(19) United States
(12) Patent Application Publication
(10) Pub. No.: US 2009/0291935 A1
(43) Pub. Date: Nov. 26, 2009

(54) X-NITRO COMPOUNDS, PHARMACEUTICAL COMPOSITIONS THEREOF AND USES THEREOF

(75) Inventors: Mark D. BEDNARSKI, Los Altos, CA (US); Lynn M. Oehler, Los Altos, CA (US); Andrew C. Haaland, Park City, UT (US); Susan J. Knox, Stanford, CA (US)

Correspondence Address: MORGAN, LEWIS & BOCKIUS, LLP, ONE MARKET SPEAR STREET TOWER, SAN FRANCISCO, CA, 94105, US

(73) Assignee: RadioRx, Inc., Menlo Park, CA (US)

(21) Appl. No.: 12/429,093

(22) Filed: Apr. 23, 2009

Related U.S. Application Data
(63) Continuation of application No. 10/681,855, filed on Oct. 7, 2003, now abandoned.
(60) Provisional application No. 60/416,936, filed on Oct. 7, 2002.
(60) Provisional application No. 60/464,782, filed on Apr. 22, 2003.

(51) Int. Cl. A61K 31/397

(52) U.S. Cl. 514/210.01

(57) ABSTRACT

The present invention provides X-nitro compound, pharmaceutical compositions of X-nitro compounds and methods of using X-nitro compounds and/or pharmaceutical compositions thereof to treat or prevent diseases or disorders characterized by abnormal cell proliferation, such as cancer, inflammation, cardiovascular disease and autoimmune disease.

The present application claims priority under 35 U.S.C. § 119(e) to U.S. Provisional Application Nos. 60/416,936 and 60/464,782, filed Oct. 7, 2002 and Apr. 22, 2003, respectively.

### 1. FIELD OF THE INVENTION

The present invention relates generally to pharmaceutical compositions of X-nitro compounds and methods of using X-nitro compounds and pharmaceutical compositions thereof to treat or prevent diseases characterized by abnormal cell proliferation such as cancer.

### 2. BACKGROUND OF THE INVENTION

Abnormal cell proliferation is a characteristic symptom of cancer. Further, abnormal cell proliferation has been implicated in numerous other diseases (e.g., cardiovascular diseases, inflammatory diseases such as rheumatoid arthritis, diabetic retinopathy, etc.). Although many methods for treating or preventing aberrant cell proliferation have been developed, a significant problem with most existing therapies is selectively distinguishing between normal and abnormal cell proliferation.

Radiotherapy is one promising approach to selectively targeting abnormal cell proliferation. A number of different radiosensitizers have been described in the art and include thiols, nitroimidazoles and metal texaphyrin compounds (See e.g., Rosenthal et al., Clin. Cancer. Res., 1999, 739). Significant problems with existing radiosensitization approaches are (1) the formation of toxic byproducts derived from the radiosensitizers, which has limited their usefulness in cancer therapy; and (2) achieving sufficiently high density of free radicals to be efficacious under dose limiting toxicity.

Another popular approach to selectively targeting abnormal cell proliferation, is treatment with bioreductive compounds, which are selectively activated in a reducing environment. Since many cancers typically contain regions of low oxygen tension (i.e., hypoxia), compounds with low redox potentials (i.e., bioreductive compounds) may be selectively activated in the reducing environment of tumor cells without external activation.

Accordingly, new compounds are required to fully explore treating or preventing abnormal cell proliferation. These new compounds may have radiotherapeutic activity or bioreductive activity. Such compounds may be effective in treating or preventing various diseases associated with abnormal cell proliferation such as cancer without forming toxic byproducts.

### 3. SUMMARY OF THE INVENTION

The present invention satisfies this and other needs by providing X-nitro compounds, pharmaceutical compositions of X-nitro compounds and methods of using X-nitro compounds or pharmaceutical compositions thereof to treat or prevent diseases associated with abnormal cell proliferation.

In a first aspect, the present invention provides methods for treating or preventing diseases or disorders characterized by abnormal cell proliferation. The methods generally involve administering to a patient in need of such treatment or prevention a therapeutically effective amount of a X-nitro compound or a pharmaceutically acceptable salt, hydrate, solvate or N-oxide thereof.

In a second aspect, the present invention provides pharmaceutical compositions of X-nitro compounds. The pharmaceutical compositions generally comprise one or more X-nitro compounds, pharmaceutically acceptable salts, hydrates, solvates or N-oxides thereof and a pharmaceutically acceptable vehicle. The choice of vehicle will depend upon, among other factors, the desired mode of administration.

In a third aspect, the current invention provides pharmaceutical compositions for treating or preventing diseases or disorders characterized by abnormal cell proliferation. The methods generally involve administering to a patient in need of such treatment or prevention a therapeutically effective amount of a pharmaceutical composition comprising a X-nitro compound or a pharmaceutically acceptable salt, hydrate, solvate or N-oxide thereof and a pharmaceutically acceptable vehicle.

### 4. DETAILED DESCRIPTION OF THE INVENTION

### 4.1 Definitions

“Pharmaceutically acceptable salt” refers to a salt of a X-nitro compound, which is pharmaceutically acceptable and possesses the desired pharmacological activity of the parent compound. Such salts: (1) acid addition salts, formed with inorganic acids such as hydrochloric acid, hydrobromic acid, sulfuric acid, nitric acid, phosphoric acid and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, t-butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by an ammonium ion, a metal ion, e.g., a alkali metal ion (e.g., sodium or potassium), an alkaline earth ion (e.g., calcium or magnesium), or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like.

“Pharmaceutically acceptable vehicle” refers to a diluent, adjuvant, excipient or carrier with which a X-nitro compound is administered.

“Patient” includes humans and other mammals.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization or eradication of a discernible symptom), physiologically, (e.g., stabilization or eradication of a physical parameter) or both. In yet another embodiment, “treating” or “treatment” refers to delaying the onset of the disease or disorder.

“Therapeutically effective amount” means the amount of a compound that, when administered to a patient for treating or preventing a disease, is sufficient to effect such treatment or prevention of the disease. The “therapeutically effective amount” will vary depending on the compound, the disease and its severity and the age, weight, etc., of the patient to be treated.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### 4.2 Methods of Using X-nitro Compounds To Treat or Prevent Abnormal Cell Proliferation

The present invention provides X-nitro compounds, pharmaceutical compositions of X-nitro compounds and methods of using X-nitro compounds or pharmaceutical compositions thereof to treat or prevent diseases associated with abnormal cell proliferation.

The methods generally involve administering to a patient in need of such treatment or prevention a therapeutically effective amount of a X-nitro compound or a pharmaceutically acceptable salt, hydrate, solvate or N-oxide thereof. In one embodiment, the X-nitro compound is intracellularly activated by the reducing environment of a tumor cell. In another embodiments, the patient is irradiated to activate the X-nitro compound. Without wishing to be bound by theory, irradiation or reduction of X-nitro compounds may lead to formation of free radicals that subsequently prevent cell replication and kill cells, presumably by interfering with DNA replication and/or reacting with cell membranes. However, other mechanisms, presently unknown, may account for the efficacy of X-nitro compounds in treating or preventing abnormal cell proliferation.

Accordingly, in some embodiments, the X-nitro compounds of the present invention may be activated by both intracellular reduction and external irradiation. In these embodiments, a synergistic or additive effect may be observed.

X-nitro compounds are generally organic compounds substituted with one or more nitro groups (i.e., nitro compounds) but also include nitrate salts (e.g., ammonium dinitride, aluminum trinitride, etc.). Typically, X-nitro compounds have a high enthalapy of formation (i.e., decomposition of X-nitro compounds releases a high amount of energy). Preferably, X-nitro compounds have an enthalapy of formation that varies between about 5 kcal/mole and about 150 kcal/mole, more preferably, between about 10 kcal/mole and about 110 kcal/mole. The enthalapy of formation of nitro compounds may be readily calculated by methods known to the skilled artisan. Accordingly, X-nitro compounds include those nitro compounds that decompose with explosive force upon activation (e.g., nitroglycerin, trinitrotoluene, trinitrobenzene, etc.). Such compounds may be readily identified by those of skill in the art by calculation of the enthalapy of formation.

X-nitro compounds may also be reduced at low reduction potentials. Cyclic voltametry demonstrates that electron transfer to X-nitro compounds occurs between about −0.1 volts and about −1.0 volts using standard electrodes (e.g., mercury or carbon cathode and platinum anode) and electrolyte solutions

X-nitro compounds include compounds where the nitro group is bonded to a carbon atom to form a nitrocarbon, to a nitrogen atom to form a nitroamine, to a sulfur atom or to a phosphorus atom and any combination thereof (e.g., in compounds that contain more then one nitro group). Accordingly, it should be understood that the present invention includes compounds where nitro groups are bonded to only one type of atom (e.g., nitrocarbons or nitroamines) as well as those compounds where nitro groups are bonded to more than one type of atom (e.g., a compound which contains a nitro group bonded to a carbon atom and a nitro group bonded to an nitrogen atom). In one embodiment, the X-nitro compound is a nitrocarbon. In another embodiment, the X-nitro compound is a nitroamine.

Preferably, X-nitro compounds contain a high density of nitro groups (i.e., the nitro groups represent a significant fraction of the overall mass of the compound). Preferably, X-nitro compounds contain two nitro groups, more preferably, three nitro groups and even more preferably, three or more nitro groups. In one embodiment, a X-nitro compound contains six nitro groups.

In one embodiment, the X-nitro compound is a nitrocarbon which has a ratio of nitro groups to carbon atoms of 1:1. In another embodiment, the X-nitro compound is a nitrocarbon which has a ratio of nitro groups to carbon atoms of 1:2.

In still another embodiment, the X-nitro compound is a nitroamine which has a ratio of nitro groups to amine nitrogen atoms of 1:1. In still another embodiment, the X-nitro compound is a nitroamine where the ratio of nitro groups to amine nitrogen atoms to carbon atoms are 1:1:1. In still another embodiment, the X-nitro compound has one nitro group bonded to every amine nitrogen atom and contains three carbon atoms and three amino nitrogen atoms. In still another embodiment, the X-nitro compound has one nitro group bonded to every amine nitrogen atom and contains four carbon atoms and three amino nitrogen atoms. In still another embodiment, the X-nitro compound has one nitro group bonded to every amine nitrogen atom and contains six carbon atoms and six amino nitrogen atoms.

Exemplary X-nitro compounds include, but are not limited to, 2,4,6,8,10,12-hexanitro-2,4,6,8,10,12-hexaazatetracyclo[5.5.0.0^{5,9}.0^{3,11}]dodecane, 1,3,5-trinitro-1,3,5-triazacyclohexane, 1,3,5,7-tetranitro-1,3,5,7 tetraazacyclooctane, 4,10-dinitro-2,6,8,12-tetraoxa-4,10-diazatetracylo[5.5.0.0^{5,9}.0^{3,11}]dodecane, 3-nitro-1,2,4-triazol-5-one, nitroguanidine, 1,3,3 trinitroazetidine, ammonium dinitride, 1,1,-diamino-2,2-dinitroethane, 2,4,6, triamino, 1,3,5 trinitrobenzene, tetranitrocarbazole and tetranitrodibenzo-1,3a,4,6a tetraazapentalene. In one embodiment, the X-nitro compound is 2, 4, 6, 8, 10,12-hexanitro-2,4,6,8,10,12-hexaazatetracyclo[5.5.0.0^{5,9}.0^{3,11}]dodecane, 1,3,5-trinitro-1,3,5-triazacyclohexane or 1,3,5,7-tetranitro-1,3,5,7 tetraazacyclooctane.

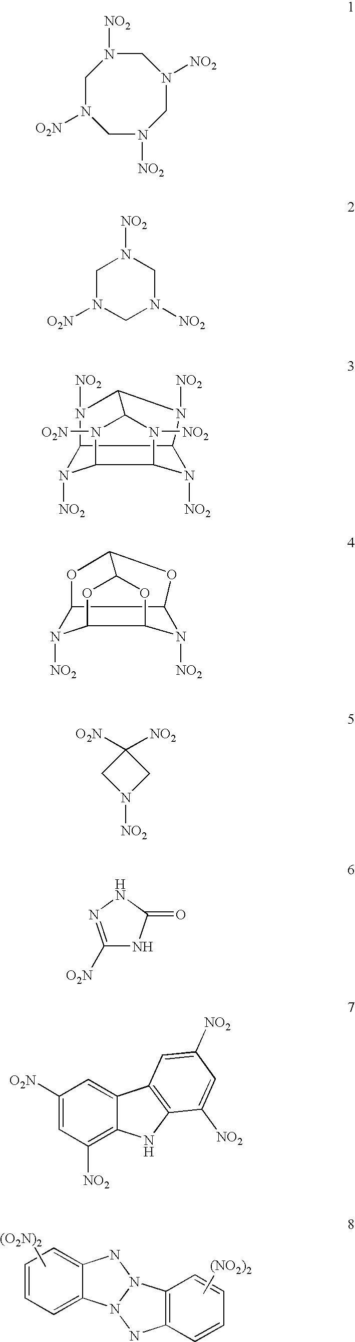

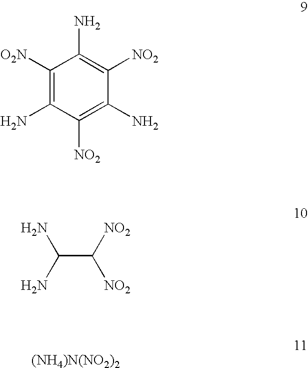

Shown above are structures for some exemplary X-nitro compounds, which may be used in the current invention.

X-nitro compounds may exist in several tautomeric forms and mixtures thereof. X-nitro compounds may also include isotopically labeled compounds where one or more atoms have an atomic mass different from the atomic mass conventionally found in nature. Examples of isotopes that may be incorporated into X-nitro compounds include, but are not limited to, ²H, ³H, ¹³C, ¹⁴C, ¹⁵N, ¹⁸O and ¹⁷O. X-nitro compounds may exist in unsolvated forms as well as solvated forms, including hydrated forms or a N-oxides. In general, hydrated and solvated forms are within the scope of the present invention. Certain X-nitro compounds may exist in multiple crystalline or amorphous forms. In general, all physical forms are equivalent for the uses contemplated by the present invention and are intended to be within the scope of the present invention.

X-nitro compounds may be activated by intracellular reduction. In one embodiment, X-nitro compounds are activated by intracellular reduction in hypoxic tumor cells, secondary to elevated glutathione levels (high GSH:GSSG (i.e., glutathione to glutathione disulfide ratios)) and possibly high levels of other antioxidant enzymes in many tumor cells and/or a median tumor cell pO₂ of less than about 10 mm Hg.

X-nitro compounds may also be activated by application of external energy. Methods useful for decomposing X-nitro compounds include, but are not limited to, irradiation (e.g., with x-rays, visible light, infrared irradiation) ultrasound (e.g. focused ultrasound), electrochemical reduction, heating, co-administration of free radical initiators (e.g., thiols), etc. In one embodiment, a X-nitro compound is activated by photon irradiation of the patient. Preferably, the patient's tumor is irradiated using a linear accelerator at a dose rate of about 100 cGy/min. The patient may also be treated with electron beam therapy, interoperative radiation therapy, stereostatic radiosurgery and high or low dose brachytherapy.

In some situations the entire patient may be irradiated. More preferably, a portion of the patient is irradiated so that only X-nitro compound localized in the irradiated portion (e.g., tumor region) of the patient is activated. Preferably, the portion of the patient which is irradiated is the site of abnormal cell proliferation.

X-nitro compounds may be obtained via conventional synthetic methods described in the art or are commercially available, e.g., from ATK Thiokol, Salt Lake City, Utah. Starting materials useful for preparing X-nitro compounds and intermediates thereof are commercially available or can be prepared by well-known synthetic methods. Other methods for synthesis of the X-nitro compounds described herein and/or starting materials are either described in the art or will be readily apparent to the skilled artisan.

In accordance with the invention, a X-nitro compound or a pharmaceutical composition thereof is administered to a patient, preferably a human, suffering from a disease characterized by abnormal cell proliferation. The X-nitro compound and pharmaceutical compositions thereof may be used to treat or prevent diseases characterized by abnormal cell proliferation.

Preferably, diseases characterized by abnormal cell proliferation include cancer (e.g., any vascularized tumor, preferably, a solid tumor, including but not limited to, carcinomas of the lung, breast, ovary, stomach, pancreas, larynx, esophagus, testes, liver, parotid, bilary tract, colon, rectum, cervix, uterus, endometrium, kidney, bladder, prostrate, thyroid, squamous cell carcinomas, adenocarcinomas, small cell carcinomas, melanomas, gliomas, neuroblastomas, sarcomas (e.g., angiosarcomas, chondrosarcomas), diabetes, cardiovascular diseases (e.g., arteriosclerosis), inflammatory diseases (e.g., arthritis, diabetic retinopathy, rheumatoid arthritis, neovascular glaucoma and psoriasis) and autoimmune diseases.

In another embodiment, X-nitro compounds may be used for in-vitro sterilization. Biological solutions may be treated with X-nitro compounds, which are toxic to pathogenic bacteria, viruses and cells. This process can also be catalyzed by the application of external energy such as light and heat.

Further, in certain embodiments, a X-nitro compound and/or pharmaceutical compositions thereof are administered to a patient, preferably a human, as a preventative measure against various diseases or disorders characterized by abnormal cell proliferation. Thus, X-nitro compounds and/or pharmaceutical compositions thereof may be administered as a preventative measure to a patient having a predisposition for a disease characterized by abnormal cell proliferation. Accordingly, X-nitro compounds and/or pharmaceutical compositions thereof may be used for the prevention of one disease or disorder and concurrently treating another (e.g., preventing arthritis while treating cancer).

The suitability of X-nitro compounds and/or pharmaceutical compositions thereof in treating or preventing various diseases or disorders characterized by abnormal cell proliferation may be determined by methods described herein (see Examples 1-7) and in the art. Accordingly, it is well with the capability of those of skill in the art to assay and use X-nitro compounds and/or pharmaceutical compositions thereof to treat or prevent diseases characterized by abnormal cell proliferation.

### 4.3 Therapeutic/Prophylactic Administration

X-nitro compounds and/or pharmaceutical compositions thereof may be advantageously used in human medicine. As previously described in Section 4.2 supra, X-nitro compounds and/or pharmaceutical compositions thereof are useful for the treatment or prevention of various diseases or disorders such as those listed above.

When used to treat or prevent the above disease or disorders, X-nitro compounds and/or pharmaceutical compositions thereof may be administered or applied singly, or in combination with other agents. X-nitro compounds and/or pharmaceutical compositions thereof may also be administered or applied singly, or in combination with other pharmaceutically active agents (e.g. other anti-cancer agents, other arthritis agents, etc.), including other X-nitro compounds and/or pharmaceutical compositions thereof.

The current invention provides methods of treatment and prophylaxis by administration to a patient of a therapeutically effective amount of a X-nitro compound and/or pharmaceutical composition thereof. The patient is preferably, a mammal and most preferably, is a human.

X-nitro compounds and/or pharmaceutical compositions thereof may be administered orally. X-nitro compounds and/or pharmaceutical compositions thereof may also be administered by any other convenient route, for example, by infusion or bolus injection, by absorption through epithelial or mucocutaneous linings (e.g., oral mucosa, rectal and intestinal mucosa, etc.). Administration can be systemic or local.

Various delivery systems are known, (e.g., encapsulation in liposomes, microparticles, microcapsules, capsules, etc.) that can be used to administer a X-nitro compound and/or pharmaceutical composition thereof. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes or skin. The preferred mode of administration is left to the discretion of the practitioner, and will depend in-part upon the site of the medical condition. In most instances, administration will result in the release of X-nitro compounds and/or pharmaceutical compositions thereof into the bloodstream.

In specific embodiments, it may be desirable to administer one or more X-nitro compounds and/or pharmaceutical compositions thereof locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes or fibers. In one embodiment, administration can be by direct injection at the site (or former site) of the disease or disorder.

In certain embodiments, it may be desirable to introduce one or more X-nitro compounds and/or pharmaceutical compositions thereof into the central nervous system by any suitable route, including intraventricular, intrathecal and epidural injection. Intraventricular injection may be facilitated by an intraventricular catheter, for example, attached to a reservoir, such as an Ommaya reservoir.

X-nitro compounds and/or pharmaceutical compositions thereof may also be administered directly to the lung by inhalation. For administration by inhalation, X-nitro compounds and/or pharmaceutical composition thereof may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver X-nitro compounds and/or pharmaceutical compositions thereof directly to the lung.

Alternatively, a Dry Powder Inhaler (“DPI”) device may be used to administer a X-nitro compound and/or pharmaceutical composition thereof to the lung. DPI devices typically use a mechanism such as a burst of gas to create a cloud of dry powder inside a container, which may then be inhaled by the patient and are well known in the art. A popular variation is the multiple dose DPI (“MDDPI”) system, which allows for the delivery of more than one therapeutic dose. MDDPI devices are commercially available from a number of pharmaceutical companies e.g., Schering Plough, Madison, N.J.). For example, capsules and cartridges of gelatin for use in an inhaler or insufflator may be formulated containing a powder mix of a X-nitro compound and/or pharmaceutical composition thereof and a suitable powder base such as lactose or starch for these systems.

Another type of device that may be used to deliver a X-nitro compound and/or pharmaceutical composition thereof to the lung is a liquid spray device supplied, for example, by Aradigm Corporation, Hayward, Calif. Liquid spray systems use extremely small nozzle holes to aerosolize liquid drug formulations that may then be directly inhaled into the lung.

In one embodiment, a nebulizer is used to deliver a X-nitro compound and/or pharmaceutical composition thereof to the lung. Nebulizers create aerosols from liquid drug formulations by using, for example, ultrasonic energy to form fine particles that may be readily inhaled (see e.g., Verschoyle et al., British J Cancer, 1999, 80, Suppl. 2, 96). Examples of nebulizers include devices supplied by Sheffield Pharmaceuticals, St. Louis, Mo. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, Ohio).

In another embodiment, an electrohydrodynamic (“EHD”) aerosol device is used to deliver a X-nitro compound and/or pharmaceutical composition thereof to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering a X-nitro compound and/or pharmaceutical composition thereof to the lung with an EHD aerosol device and such optimization is routinely performed by one of skill in the art. EHD aerosol devices may more efficiently deliver drugs to the lung than existing pulmonary delivery technologies.

In another embodiment, a X-nitro compound and/or pharmaceutical compositions thereof can be delivered in a vesicle, in particular a liposome (e.g., Langer, 1990, Science, 249:1527-1533; Treat et al., in “Liposomes in the Therapy of Infectious Disease and Cancer,” Lopez-Berestein and Fidler (eds.), Liss, New York, pp. 353-365 (1989)).

In another embodiment, a X-nitro compound and/or pharmaceutical compositions thereof can be delivered via sustained release systems, preferably oral sustained release systems. In one embodiment, a pump may be used (e.g., Langer, supra, Sefton, 1987, CRC Crit. Ref Biomed. Eng. 14:201; Saudek et al., 1989, N. Engl. J. Med. 321:574).

In another embodiment, polymeric materials can be used (e.g., “Medical Applications of Controlled Release,” Langer and Wise (eds.), CRC Press, Boca Raton, Fla. (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228: 190; During et al., 1989, Ann. Neurol. 25:351; Howard et al., 1989, J. Neurosurg. 71:105).

In another embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Pharm. Tech. & Prod. Mfr. 1984, 5 (3) 1-9). Factors affecting drug release are well known to the skilled artisan and have been described in the art (Bamba et al., Int. J. Pharm. 1979, 2, 307).

In another embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In another embodiment, OROS™ osmotic devices are used for oral sustained release delivery devices (Theeuwes et al., U.S. Pat. No. 3,845,770; Theeuwes et al., U.S. Pat. No. 3,916,899).

In yet another embodiment, a controlled-release system can be placed in proximity of the target of the X-nitro compound and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

### 4.4 Pharmaceutical Compositions

The present pharmaceutical compositions typically contain a therapeutically effective amount of one or X-nitro compounds, preferably, in purified form, together with a suitable amount of a pharmaceutically acceptable vehicle, so as to provide the form for proper administration to a patient. When administered to a patient, the X-nitro compound and pharmaceutically acceptable vehicles are preferably sterile. Water is a preferred vehicle when the X-nitro compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a X-nitro compound may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (e.g., Grosswald et al., U.S. Pat. No. 5,698,155). A general discussion of the preparation of pharmaceutical compositions may be found in Remington, “The Science and Practice of Pharmacy,” 19^{th} Edition.

For topical administration a X-nitro compound may be formulated as solutions, gels, ointments, creams, suspensions, etc. as is well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In a preferred embodiment, X-nitro compounds are formulated in accordance with routine procedures as a pharmaceutical composition adapted for intravenous administration to human beings. Typically, X-nitro compounds are solutions in sterile isotonic aqueous buffer for intravenous administration. For injection, X-nitro compounds may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent. Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When the X-nitro compounds are administered by infusion, it can be dispensed, for example, with an infusion bottle containing sterile pharmaceutical grade water or saline. When the X-nitro compound is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optional agents, for example, sweetening agents such as fructose, aspartame or saccharin; flavoring agents such as peppermint, oil of wintergreen, or cherry coloring agents and preserving agents, to provide a pharmaceutically palatable preparation. Moreover, when in tablet or pill form, the pharmaceutical compositions may be coated to delay disintegration and absorption in the gastrointestinal tract, thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM), etc. Additionally, flavoring agents, preservatives, coloring agents, bile salts, acylcamitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices typically include a X-nitro compound with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A X-nitro compound may also be formulated in rectal or vaginal pharmaceutical compositions such as suppositories or retention enemas, e.g., containing conventional suppository bases such as cocoa butter or other glycerides.

In addition to the formulations described previously, a X-nitro compound may also be formulated as a depot preparation. Such long acting formulations may be administered by implantation (for example, subcutaneously or intramuscularly) or by intramuscular injection. Thus, for example, a X-nitro compound may be formulated with suitable polymeric or hydrophobic materials (e.g., as an emulsion in an acceptable oil) or ion exchange resins, or as sparingly soluble derivatives, such as a sparingly soluble salt.

When a X-nitro compound is acidic or basic, it may be included in any of the above-described formulations as the free acid or free base, a pharmaceutically acceptable salt, a solvate or hydrate. Pharmaceutically acceptable salts substantially retain the activity of the free acid or base, may be prepared by reaction with bases or acids and tend to be more soluble in aqueous and other protic solvents than the corresponding free acid or base form.

### 4.5 Doses

A X-nitro compound and/or pharmaceutical composition thereof, will generally be used in an amount effective to achieve the intended purpose. For use to treat or prevent the above diseases or disorders the X-nitro compound and/or pharmaceutical compositions thereof, are administered or applied in a therapeutically effective amount.

The amount of a X-nitro compound and/or pharmaceutical composition thereof that will be effective in the treatment of a particular disorder or condition disclosed herein will depend on the nature of the disorder or condition, and can be determined by standard clinical techniques known in the art. In addition, in vitro or in vivo assays may optionally be employed to help identify optimal dosage ranges. The amount of a X-nitro compound and/or pharmaceutical composition thereof administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. Dosing may be repeated intermittently, may be provided alone or in combination with other drugs and may continue as long as required for effective treatment of the disease state or disorder.

Suitable dosage ranges for oral administration are dependent on the efficiency of radiosensitization, but are generally about 0.001 mg to about 100 mg of the X-nitro compound per kg body weight. Dosage ranges may be readily determined by methods known to the artisan of ordinary skill.

Suitable dosage ranges for intravenous (i.v.) administration are about 0.01 mg to about 100 mg per kg/body weight. Suitable dosage ranges for intranasal administration are generally about 0.01 mg/kg body weight to about 1 mg/kg body weight. Suppositories generally contain about 0.01 milligram to about 50 milligrams of a X-nitro compound per kg/body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kg/body weight. Effective doses may be extrapolated from dose-response curves derived from in vitro or animal model test systems. Such animal models and systems are well-known in the art.

The X-nitro compounds are preferably assayed in vitro and in vivo, for the desired therapeutic or prophylactic activity, prior to use in humans. For example, in vitro assays can be used to determine whether administration of a specific X-nitro compound or a combination of X-nitro compounds is preferred. The X-nitro compound may also be demonstrated to be effective and safe using animal model systems.

Preferably, a therapeutically effective dose of a X-nitro compound and/or pharmaceutical composition thereof described herein will provide therapeutic benefit without causing substantial toxicity. Toxicity of X-nitro compounds and/or pharmaceutical compositions thereof may be determined using standard pharmaceutical procedures and may be readily ascertained by the skilled artisan. The dose ratio between toxic and therapeutic effect is the therapeutic index. A X-nitro compound and/or pharmaceutical composition thereof will preferably exhibit particularly high therapeutic indices in treating disease and disorders characterized by aberrant abnormal cell proliferation. The dosage of a X-nitro compound and/or pharmaceutical composition thereof described herein will preferably be within a range of circulating concentrations that include an effective dose with little or no toxicity.

### 4.9. Combination Therapy

In certain embodiments of the present invention, X-nitro compounds and/or pharmaceutical compositions thereof can be used in combination therapy with at least one other therapeutic agent. The X-nitro compound and/or pharmaceutical composition thereof and the therapeutic agent can act additively or, more preferably, synergistically. In one embodiment, a X-nitro compound and/or a pharmaceutical composition thereof is administered concurrently with the administration of another therapeutic agent. In another embodiment, a X-nitro compound and/or pharmaceutical composition thereof is administered prior or subsequent to administration of another therapeutic agent.

In particular, in one embodiment, X-nitro compounds and/or pharmaceutical compositions thereof can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan), nitrosoureas, triazines)), antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxurubicin, bleomycin, mithrmycin, mitomycin C, L-asparaginase, interferon alpha), platinum coordination complexes (e.g., cis-platinum, carboplatin, etc.), apoptosis inducing agents, glutathione depleting agents or other agents that can alter the redox status of the cell. Those of skill in the art will appreciate that X-nitro compounds may also be used in concurrent combination therapy with both the chemotherapeutic agents listed above and radiotherapy.

### 4.10. Therapeutic Kits

The current invention also provides therapeutic kits comprising X-nitro compounds and/or pharmaceutical compositions thereof. The therapeutic kits may also contain other compounds (e.g., chemotherapeutic agents, natural products, apoptosis-inducing agents, etc.) or pharmaceutical compositions thereof.

Therapeutic kits may have a single container which contains a X-nitro compound and/or pharmaceutical compositions thereof with or without other components (e.g., other compounds or pharmaceutical compositions of these other compounds) or may have distinct container for each component. Preferably, therapeutic kits include a X-nitro compound and/or a pharmaceutical composition thereof packaged for use in combination with the co-administration of a second compound (preferably, a chemotherapeutic agent, a natural product, an apoptosis-inducing agent, etc.) or a pharmaceutical composition thereof. The components of the kit may be pre-complexed or each component may be in a separate distinct container prior to administration to a patient.

The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid.

Preferably, a therapeutic kit will contain apparatus (e.g., one or more needles, syringes, eye droppers, pipette, etc.), which enables administration of the components of the kit.

### 5. EXAMPLES

The invention is further defined by reference to the following examples, which describe in detail, preparation of compounds and methods for assaying for biological activity. It will be apparent to those skilled in the art that many modifications, both to materials and methods, may be practiced without departing from the scope.

### 5.1 Example 1

### Initial In Vitro Experiments

2,4,6,8,10,12-hexanitro-2,4,6,8,10,12-hexaazatetracyclo[5.5.0.0^{5,9}.0^{3,11}]dodecane, 1,3,5-trinitro-1,3,5-triazacyclohexane, 1,3,5,7-tetranitro-1,3,5,7 tetraazacyclooctane, 4,10-dinitro-2,6,8,12-tetraoxa-4,10-diazaietracylo[5.5.0.0^{5,9}.0^{3,11}]dodecane, 3-nitro-1,2,4-triazol-5-one, nitroguanidine, 1,3,5-triamino-2,4,6,1,3,3 trinitroazetidine, ammonium dintride, 1,1,-diamino-2,2-dinitroethane, tetranitrocarbazole or tetranitrodibenzo-1,3a,4,6a tetraazapentalene are studied in vitro in 2-5 cell lines selected for example from Table 1, below. These cell lines are already well characterized in terms of radiation response (radiobiological parameters of radiation dose response curves) as shown in Table 1 below.

TABLE 1
Characterization of in vitro survival curves of human tumor cell lines.

| | | LQ^{a} | | | SHMT^{b} | | |
|---|---|---|---|---|---|---|---|
| Cell line | | α | β | α/β | D₀ | D_{q} | N |
| Caki-1 | Renal cell | 0.36 | 0.059 | 6.10 | 1.06 | 1.45 | 3.94 |
| A498 | Renal cell mm | 0.14 | 0.058 | 2.41 | 1.03 | 3.16 | 21.23 |
| HT29 | Colon adenocarcinoma | 0.11 | 0.039 | 2.82 | 1.25 | 3.90 | 22.59 |
| LS174T | Colon | 0.34 | 0.064 | 5.31 | 0.89 | 1.51 | 5.51 |
| | adenocarcinoma | | | | | | |
| SNB75 | Glioma | 0.05 | 0.040 | 1.25 | 1.37 | 3.78 | 16.00 |
| A549 | Lung carcinoma | 0.00 | 0.037 | — | 1.12 | 6.15 | 246.4 |
| H69 | Lung small call ca. | 0.21 | 0.06 | 3.50 | 1.75 | 1.10 | 2.38 |
| H128 | Lung small cell ca. | 0.20 | 0.13 | 1.54 | 1.04 | 1.26 | 11.45 |
| HT180 | Fibrosarcoma | 0.00 | 0.048 | — | 1.12 | 4.59 | 59.38 |
| SCC-4 | Tongue SCC | 0.30 | 0.05 | 6.00 | 1.02 | 2.08 | 7.69 |
| SCC-9 | Tongue SCC | 0.30 | 0.02 | 15.00 | 1.41 | 2.34 | 5.23 |
| SCC-15 | Tongue SCC | 0.05 | 0.13 | 0.38 | 0.91 | 1.80 | 7.23 |
| SCC-25 | Tongue SCC | 0.37 | 0.05 | 7.40 | 1.05 | 1.61 | 4.64 |
| RPMI2650 | Nasal SCC | 0.51 | 0.01 | 51.00 | 1.47 | 1.04 | 2.04 |
| FaDu | Pharynx SCC | 0.13 | 0.04 | 3.25 | 1.40 | 2.82 | 7.50 |
| Detroit562 | Pharynx ECC | 0.26 | 0.03 | 8.67 | 1.37 | 2.64 | 6.93 |
| KB | Oral cavity ECC | 0.31 | 0.03 | 10.30 | 1.36 | 2.17 | 4.94 |
| HEp-2 | Larynx ECC | 0.44 | 0.04 | 11.00 | 1.08 | 1.51 | 4.05 |
| A253 | Submaxillary gland ECC | 0.24 | 0.03 | 8.00 | 1.46 | 2.47 | 5.44 |
| HEL | Erythroid leukemia | 0.14 | 0.10 | 1.40 | 1.41 | 1.43 | 3.78 |
| Peer | T cell ALL | 0.23 | 0.32 | 0.72 | 0.65 | 0.87 | 3.82 |

^{a}The single-hit multi-target model.
^{b}The linear quadratic model.
^{c}SCC, squamous call carcinoma; ECC, epidermoid cell carcinoma.

Cell lines are irradiated using a ¹³⁷Cs source at a dose rate of 422 cGy/min with a range of radiation doses (e.g., 0, 200, 400, 600, 800, 1000, 1500 and 2000 cGy) with and without 2,4,6,8,10,12-hexanitro-2,4,6,8,10,12-hexaazatetracyclo[5.5.0.0^{5,9}.0^{3,11}]dodecane, 1,3,5-trinitro-1,3,5-triazacyclohexane, 1,3,5,7-tetranitro-1,3,5,7 tetraazacyclooctane, 4,10-dinitro-2,6,8,12-tetraoxa-4,10-diazatetracylo[5.5.0.0^{5,9}.0^{3,11}]dodecane, 3-nitro-1,2,4-triazol-5-one, nitroguanidine, 1,3,5-triamino-2,4,6,1,3,3 trinitroazetidine, ammonium dintride, 1,1,-diamino-2,2-dinitroethane, tetranitrocarbazole or tetranitrodibenzo-1,3a,4,6a tetraazapentalene at a final concentration of 1, 10, 50 and 100 mM in DMSO. The above compounds contain high density nitro groups for free radical formation upon initiation with radiation.

The following assays are performed and are well-known to the skilled artisan:
1) MTT proliferation assay;
2) Clonogenic survival assay (Rupnow et al., Cell Death Differ. 1998, 5(2): 141-147);
3) Quantitation of overall survival and apoptosis (Rupnow et al., Cell Death Differ. 1998, 5(2): 141-147; Armstrong et al., Cell Death Differ., 2002, 9(3): 252-263); and
4) Measurement of ROS (Armstrong et al., Cell Death Differ. 2002, 9(3): 252-263).

Results from the above experiments allow for assessment of radiosensitization of a variety of tumor types (cell lines) using well established methods of analysis (Ning et al., Radiat. Res. 2002, 157(1): 45-51). Then, the following in vivo experiments are performed with the most efficacious of the compounds studied.

### 5.2 Example 2

### Pilot Toxicity Study

Drug: 5 doses plus DMSO vehicle control; 6 groups total with 10 mice (C3H)/gp=60 mice
Duration: 4 weeks
Endpoints: weight loss, survival.
   necropsy of all unexplained deaths
      possible counting of blood cells (CBC) and chemical panels on a subset of animals pre tx and q week
         s

### 5.3 Example 3

### Dose Response for Drug in C3h Mice with SCCVII and Rif-1 Tumors

Drug: 4 doses, 4 groups total with 10 mice per group×2 tumor types=80 mice. Duration 8 weeks.

### 5.4 Example 4

### Drug with and without a Single Dose of Radiation at 2 Doses (5 Gy and 10 Gy) (Including Untreated Control, Drug Alone, Radiation Alone and Studies of Drug Administered Concurrently with Radiation) in 2 Models

Doses are decided by results of Example 2. 12 groups total with 10 mice per group=120 mice. Duration: 8 weeks.

### 5.5 Example 5

### Drug with and without Clinically Relevant Multiply Fractionated Radiation at 2 Doses (2 Gy and 3 Gy Daily Tx) Including Untreated Control, Drug Alone, Radiation Alone and Studies of Drug Administered Concurrently with Radiation) in 2 Models.

Doses are mutually decided at conclusion of Example 3. 12 groups total with 10 mice per group=120 once. Duration: 8 weeks.

Mouse tumors are irradiated as previously described (Ning et al., Radiat. Res. 2002, 157(1): 45-51) using a Philips RT-250 200 κVp x-ray unit (12.5 mA; half value layer of 1.0 turn Cu) at a dose rate of 1.04 Gy/min. Data from the above experiments is analyzed as previously described (Ning et al., Radiat. Res. 2002, 157(1): 45-51).

### 5.6 Example 6

### Measurement of Activation of X-Nitro Compounds in Tumor Cells

The following set of conditions may be used to manipulate the intracellular redox status of the tumor cells studied:

Normal media (control);
Buthionine sulfoxamine (BSO) at approximately 1 mM to deplete GSH;
N-Acetyl Cysteine (NAC) at 50 mM to reduce cellular ROS;
alpha-lipoic acid at 0.2 mM to increase intracellular GSH reduction;
hydrogen peroxide at 2-20 mM for oxidation; and
Xanthine/Xanthine oxidase (100 uU/ml xanthine oxidase, 1 mM xanthine) to generate ROS.

Cells are incubated with the above in 5% CO2 at 37 degrees C. for 0-72 hours prior to addition of a X-nitro compound. At time zero and immediately prior to the addition of the X-nitro compound, the GSH level and GSH/GSSG ratio are measured. Tumor cell survival (over all cell killing and apoptosis) are measured at various time points including 0,12,24,48, and 72 hours following the addition of the test compounds to the media using methods known to the skilled artisan. ROS generation is measured and correlated with the following parameters: chemical redox potential of the X-nitro compound, cellular redox status at baseline, ROS generation and cell death.

### 5.7 Example 7

### Cytotoxicity of X-Nitro Compounds Against Tumor Cells

In general cells (e.g. HT29) were grown on tissue culture plates and were used while growing in the exponential phase. Cells were treated with increasing concentration of X-nitro compound. Accordingly, cell lines were treated at a final concentration of 1, 10, 50 and 100 uM of X-nitro compound which was added to cell cultures in DMSO. The amount of cell death is measured by the MTT assay. Cell death (or survival) is plotted versus concentration of compound and an LC 50 (or LC 90) is determined by measuring the concentration at which 50% (or 90%) of the cells die. The results of the MTT cell assay were confirmed by the clonogenic survival assay (Rupnow et al., Cell Death Differ. 1998, 5(2): 141-147). The LC 50 of 2,4,6,8,10,12-hexanitro-2,4,6,8,10,12-hexaazatetracyclo[5.5.0.0^{5,9}.0^{3,11}]dodecane, 1,3,5-trinitro-1,3,5-triazacyclohexane, 1,3,5,7-tetranitro-1,3,5,7 tetraazacyclooctane, 4,10-dinitro-2,6,8,12-tetraoxa-4,10-diazatetracylo[5.5.0.0^{5,9}.0^{3,11}]dodecane, 3-nitro-1,2,4-triazol-5-one and 1,3,3 trinitroazetidine ranged between about 5.0 mM and 20 and about μM.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference.

All references and publications cited herein are incorporated by reference in their entirety.

What is claimed is:

1-31. (canceled)

32. A pharmaceutical formulation comprising a compound having the formula: 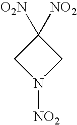
and a pharmaceutically acceptable vehicle.

33. The pharmaceutical formulation according to claim 32 wherein said pharmaceutical formulation is a unit dosage formulation comprising a therapeutically effective amount of said compound.

34. The pharmaceutical formulation according to claim 32 wherein said formulation is an oral dosage form and said therapeutically effective amount is from about 0.001 mg/kg body weight to about 100 mg/kg of body weight.

35. The pharmaceutical formulation according to claim 32 wherein said formulation is an intravenous dosage form and said therapeutically effective amount is from about 0.01 mg/kg body weight to about 100 mg/kg of body weight.

36. The pharmaceutical formulation according to claim 32 wherein said vehicle is an aqueous vehicle.

37. The pharmaceutical formulation according to claim 36 wherein said vehicle is sterile, isotonic aqueous buffer.

38. The pharmaceutical formulation according to claim 36 further comprising a member selected from a suspending agent, a stabilizing agent, a dispersing agent and a solubilizing agent.

39. An infusion bottle containing a member selected from sterile pharmaceutical grade water and saline, and further comprising a compound having the formula:

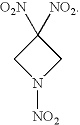

40. A container comprising a unit dosage formulation of a compound having the formula: 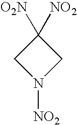
as a member selected from a lyophilized powder and a water free concentrate.
